(19) United States
(12) Patent Application Publication
(10) Pub. No.: US 2022/0101989 A1
(43) Pub. Date: Mar. 31, 2022

(54) CUSTOMIZED MEDICAL TREATMENT

(71) Applicant: GoForward, Inc., San Francisco, CA (US)

(72) Inventors: Adrian Aoun, San Francisco, CA (US); Casey Edgeton, San Francisco, CA (US); Chandrashekar Raghavan, Alameda, CA (US); Paras Parimal Jhaveri, San Francisco, CA (US); Erik Lauri Frey, Palo Alto, CA (US); Eugene Leonard Huang, San Francisco, CA (US); James Francis Hamlin, San Francisco, CA (US); Charles Chu Chen, San Francisco, CA (US); Shradda Rakesh Bagrodia, San Francisco, CA (US); Isaac Daniel Gateno, San Francisco, CA (US)

(21) Appl. No.: 17/482,037

(22) Filed: Sep. 22, 2021

Related U.S. Application Data
(60) Provisional application No. 63/083,416, filed on Sep. 25, 2020.

(51) Int. Cl. G16H 40/20; G16H 10/60; G16H 50/70; G16H 50/20; G16H 50/30; G16H 40/67; A61B 5/00

(52) U.S. Cl. CPC G16H 40/20; G16H 10/60; G16H 50/70; G16H 10/40; G16H 50/30; G16H 40/67; A61B 5/7282; G16H 50/20

(57) ABSTRACT

Systems, methods, and non-transitory media are included for a providing a customized medical treatment. An example method can include receiving, at a medical system, patient data associated with a patient; detecting, at the medical system, a health condition of the patient based on the patient data associated with the patient; comparing, at the medical system, the patient data and the health condition of the patient with similar health conditions of a general population; determining, at the medical system, whether the health condition of the patient is actionable based on the comparing of the patient data and the health condition of the patient with the similar health conditions of the general population; and providing, by the medical system, a customized medical treatment plan to the patient based on the determining of whether the health condition of the patient is actionable.

### CROSS-REFERENCE TO RELATED APPLICATIONS

This application claims the benefit of priority of U.S. Provisional Application No. 63/083,416, filed on Sep. 25, 2020, entitled “CUSTOMIZED MEDICAL TREATMENT”, the contents of which are incorporated by reference in their entirety and for all purposes.

### TECHNICAL FIELD

The present disclosure generally relates to providing customized medical treatment for patients.

### BACKGROUND

Health care providers use a variety of tools to provide patient care and consultations, such as health records and medical systems. In general, a patient visits a provider's office to seek a medical consultation, treatment, procedure, and care. During the patient's visit, the provider can examine the patient, issue a diagnosis, provide any treatments deemed necessary, perform any procedures deemed necessary, order tests, and prescribe medications or medical devices, among other medical tasks. The provider typically relies on information about the patient maintained in a medical system that may also be used to track patient information and provide care.

However, the information available in medical systems is often incomplete, inaccurate, and/or outdated. Moreover, medical systems used to track and access information about the patient for patient care and consultations are inefficient and generally lack an ability to collect medically-relevant data about the patient from distributed sources. Typically, most or all of the information in the medical systems is manually entered into the system and thus prone to errors or subject to missing information, and as a result such information is difficult to accurately maintain and update. Consequently, medical systems are significantly limited and can become unreliable over time.

### BRIEF SUMMARY

Disclosed are systems, methods, and computer-readable media for providing customized medical treatments. According to at least one example, a method is included for providing customized medical treatments. The method can include receiving, at a medical system, patient data associated with a patient; detecting, at the medical system, a health condition of the patient based on the patient data associated with the patient; comparing, at the medical system, the patient data and the health condition of the patient with similar health conditions of a general population; determining, at the medical system, whether the health condition of the patient is actionable based on the comparing of the patient data and the health condition of the patient with the similar health conditions of the general population; and providing, by the medical system, a customized medical treatment plan to the patient based on the determining of whether the health condition of the patient is actionable.

According to at least one example, an apparatus is included for providing customized medical treatments. In some examples, the apparatus can include memory and one or more processors coupled to the memory, the one or more processors being configured to receive, at a medical system, patient data associated with a patient; detect, at the medical system, a health condition of the patient based on the patient data associated with the patient; compare, at the medical system, the patient data and the health condition of the patient with similar health conditions of a general population; determine, at the medical system, whether the health condition of the patient is actionable based on the comparison of the patient data and the health condition of the patient with the similar health conditions of the general population; and provide, by the medical system, a customized medical treatment plan to the patient based on the determination of whether the health condition of the patient is actionable.

According to at least one example, a non-transitory computer-readable medium is included for providing customized medical treatments. The non-transitory computer-readable medium can include receiving, at a medical system, patient data associated with a patient; detecting, at the medical system, a health condition of the patient based on the patient data associated with the patient; comparing, at the medical system, the patient data and the health condition of the patient with similar health conditions of a general population; determining, at the medical system, whether the health condition of the patient is actionable based on the comparing of the patient data and the health condition of the patient with the similar health conditions of the general population; and providing, by the medical system, a customized medical treatment plan to the patient based on the determining of whether the health condition of the patient is actionable.

In some aspects, the method, apparatuses, and non-transitory computer-readable storage medium described above can include the patient data associated with the patient as comprising at least one of performing a medical test, performing a medical examination, and measuring a health metric via one or more medical devices. In some examples, the medical test can include at least one of a blood test, a scan, collecting and analyzing a specimen from the patient, a medical assessment, a genetic test, and a breathing test. In other examples, the health metric can include at least one of blood pressure, blood glucose levels, a pulse, a body temperature, and a body weight. In some instances, at least part of the patient data can be received from at least one of a client device associated with the patient and one or more sensors at a medical care site. In some instances, the client device can comprise at least one of a smart phone and a smart wearable device. In other instances, the one or more sensors can comprise at least one of a wireless blood pressure sensor, a wireless heart rate sensor, a wireless body temperature sensor, a wireless pulse oximeter, a stethoscope, and an imaging sensor.

In some aspects, the method, apparatuses, and non-transitory computer-readable storage medium described above can include determining an additional portion of patient data, the additional portion of patient data being based on a current context of a patient consultation; and comparing the additional portion of patient data with the patient data and the health condition of the patient with the similar health conditions of the general population.

In some aspects, the method, apparatuses, and non-transitory computer-readable storage medium described above can include providing, by the medical system, one or more workflow items determined at a patient consultation, the one or more workflow items being based on at least one of the patient data and additional patient data collected during the patient consultation.

This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings, and each claim.

The foregoing, together with other features and embodiments, will become more apparent upon referring to the following specification, claims, and accompanying drawings.

### BRIEF DESCRIPTION OF THE DRAWINGS

In order to describe the manner in which the various advantages and features of the disclosure can be obtained, a more particular description of the principles described above will be rendered by reference to specific embodiments thereof, which are illustrated in the appended drawings. Understanding that these drawings depict only example embodiments of the disclosure and are not to be considered to limit its scope, the principles herein are described and explained with additional specificity and detail through the use of the drawings in which:

FIG. 1 is a diagram illustrating an example system environment for patient care, in accordance with some examples of the present disclosure;

FIG. 2 is a diagram illustrating an example configuration of a collaborative smart screen, in accordance with some examples of the present disclosure;

FIG. 3 is a diagram illustrating an example configuration of a medical system receiving data from offsite, a consultation visit, and a medical care site, in accordance with some examples of the present disclosure;

FIG. 4 is a diagram illustrating an example process of providing a customized medical treatment, in accordance with some examples of the present disclosure;

FIG. 5 is a diagram illustrating example process of providing a customized skin condition treatment recommendation, in accordance with some examples of the present disclosure;

FIG. 6 is a flowchart illustrating an example method for providing a customized medical treatment to a patient, in accordance with some examples of the present disclosure; and

FIG. 7 illustrates an example computing device architecture, in accordance with some examples of the present disclosure.

### DETAILED DESCRIPTION

Certain aspects and embodiments of this disclosure are provided below. Some of these aspects and embodiments may be applied independently and some of them may be applied in combination as would be apparent to those of skill in the art. In the following description, for the purposes of explanation, specific details are set forth in order to provide a thorough understanding of embodiments of the application. However, it will be apparent that various embodiments may be practiced without these specific details. The figures and description are not intended to be restrictive.

The ensuing description provides example embodiments only, and is not intended to limit the scope, applicability, or configuration of the disclosure. Rather, the ensuing description of the exemplary embodiments will provide those skilled in the art with an enabling description for implementing an exemplary embodiment. It should be understood that various changes may be made in the function and arrangement of elements without departing from the spirit and scope of the application as set forth in the appended claims.

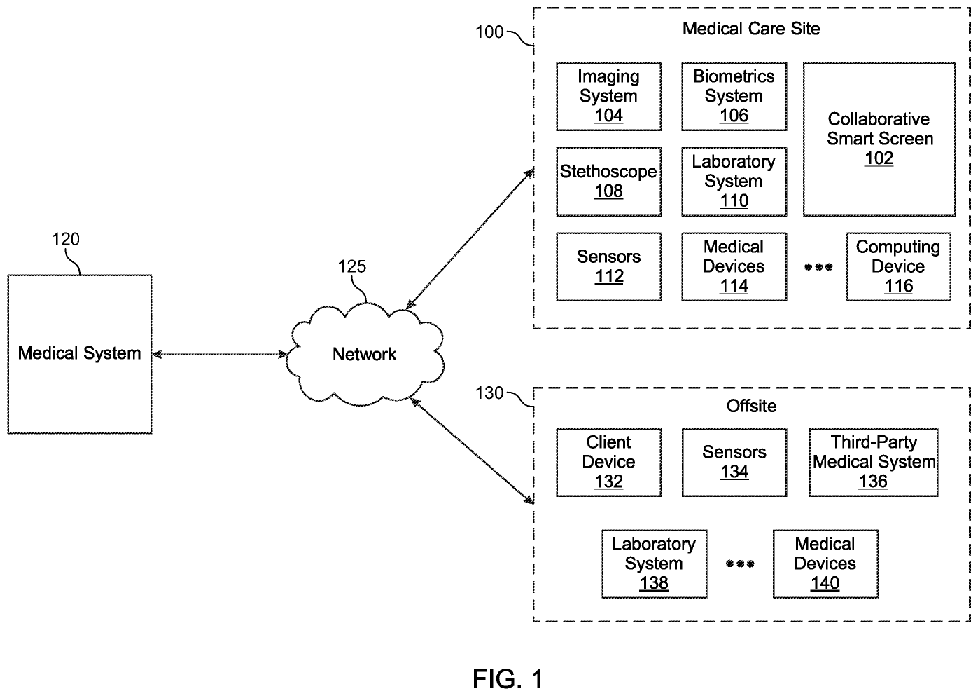

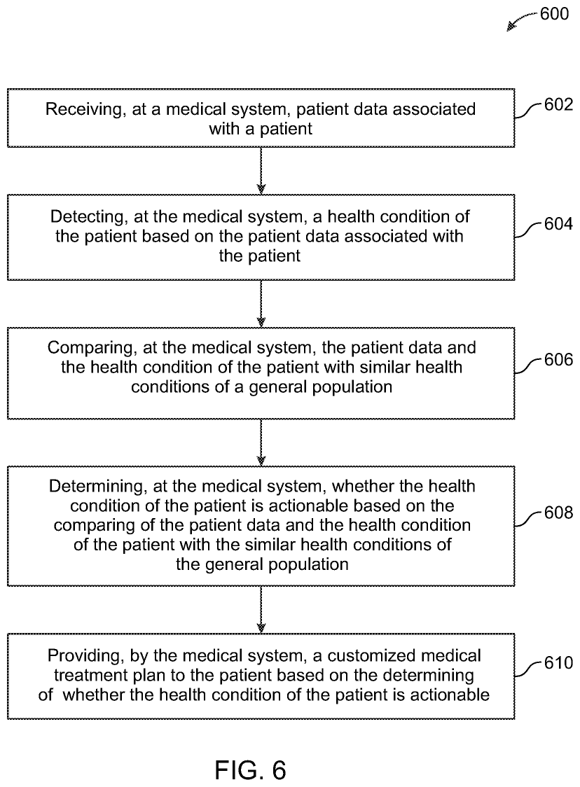

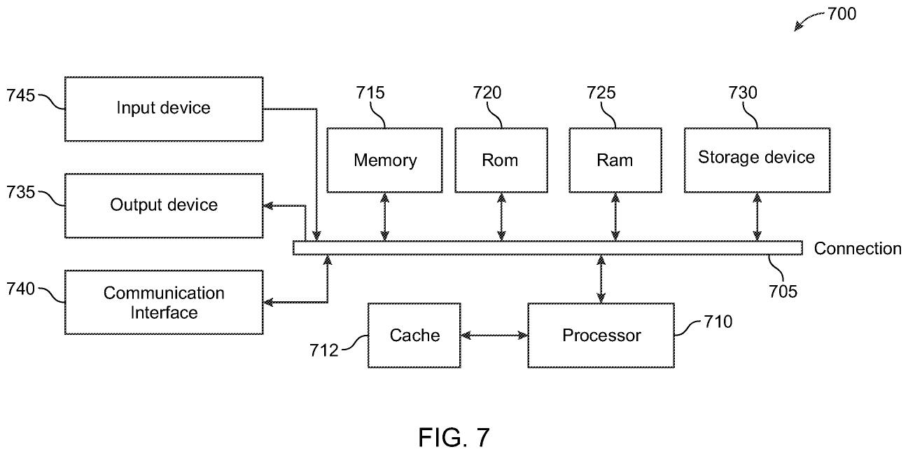

The present disclosure describes systems, methods, and computer-readable media for providing customized medical treatments. The present technologies will be described in the following disclosure as follows. The discussion begins with a description of example systems, environments and technologies for providing medical care by implementing customized medical treatments, as illustrated in FIG. 1 through FIG. 5. A description of an example method for providing customized medical treatments, as illustrated in FIG. 6, will then follow. The discussion concludes with a description of an example computing device architecture including example hardware components suitable for implementing medical systems, collaborative smart screens, and devices, as illustrated in FIG. 7. The disclosure now turns to FIG. 1.

FIG. 1 is a diagram illustrating an example system environment for patient care. In this example, the system environment includes a medical system 120, a set of devices 102-116 in a medical care site 100, and a set of devices 132-140 at one or more offsite locations 130. However, the system environment shown in FIG. 1 is merely an illustrative example provided for explanation purposes. It should be understood that, in other examples, the system environment can include more, less, and/or different systems, devices, entities, and/or sites than those shown in FIG. 1.

The medical system 120 can include one or more computing components for storing, collecting, tracking, and/or monitoring health information associated with patients. For example, the medical system 120 can include one or more computing components for storing health records, collecting health records and/or associated data and updates, providing and/or displaying health records and/or associated data, managing/maintaining scheduling information, providing notifications, providing medical requests and/or orders/prescriptions, managing health plans, etc. The one or more computing components associated with the medical system 120 can include, for example and without limitation, one or more servers, databases, storage systems, virtual machines, software containers, datacenters, data stores, computing resources, serverless functions, cloud infrastructure, computing devices, and/or any other computing resources and/or electronic devices.

In some cases, the medical system 120 can be located/hosted at the medical care site 100. In other cases, the medical system 120 can be located/hosted at a separate location or site. For example, the medical system 120 can be located/hosted at a separate medical care site, a location from the one or more offsite locations 130, on a cloud network, and/or on any other location.

The devices 102-116 in the medical care site 100 can include sensors and/or systems for collecting health metrics and/or performing medical tests or procedures. In FIG. 1, devices 102-116 in the medical care site 100 include a collaborative smart screen 102, one or more imaging systems 104, one or more biometric systems 106, one or more stethoscopes 108, one or more laboratory systems 110, one or more sensors 112, one or more medical devices 114, and one or more computing devices 116; and the devices 132-140 at the one or more offsite locations 130 can include one or more client devices 132, one or more sensors 134, one or more third-party medical systems 136, one or more laboratory systems 138, and one or more medical devices 140.

In some examples, the collaborative smart screen 102 in the medical care site 100 can include a smart, interactive system for dynamically displaying and providing medical information, including contextually relevant information as further described herein. The collaborative smart screen 102 can include one or more communication interfaces (e.g., wired and/or wireless) for communicating with other devices such as the medical system 120, and/or any other device

The one or more imaging systems 104 in the medical care site 100 can include one or more medical imaging and/or scanning systems such as, for example, an ultrasound system, an electrocardiogram device (ECG), a magnetic resonance imaging instrument (MRI), a computerized tomography (CT) scanner, a positron emission tomography (PET) scanner, a photoacoustic imaging device, a camera device, and/or any other imaging and/or scanning device. The one or more imaging systems 104 can include one or more communication interfaces (e.g., wired and/or wireless) for communicating test results and/or measurements to other devices such as the medical system 120, the collaborative smart screen 102, and/or any other device.

The one or more biometrics systems 106 in the medical care site 100 can include one or more biometrics sensors and/or devices such as, for example, a heart rate sensor, a blood pressure sensor, a temperature sensor, a pulse oximeter, a blood glucose sensor, a weight scale, a body composition machine/analyzer, and/or any other sensor or system for measuring biometrics. The one or more biometric systems 106 can include one or more communication interfaces (e.g., wired and/or wireless) for communicating test results and/or measurements to other devices such as the medical system 120, the collaborative smart screen 102, and/or any other device.

The one or more stethoscopes 108 in the medical care site 100 can include an electronic stethoscope. In some examples, the electronic stethoscope can include a wireless stethoscope capable of wirelessly communicating with other devices and providing measurements. The one or more stethoscopes 108 can include one or more communication interfaces (e.g., wired and/or wireless) for communicating test results and/or measurements to other devices such as the medical system 120, the collaborative smart screen 102, and/or any other device.

The one or more laboratory systems 110 and 138 can include laboratory equipment, one or more tools, and/or one or more devices for collecting, analyzing, and/or interpreting specimens such as, for example, blood samples, saliva, stool samples, urine, skin samples, etc. The one or more laboratory systems 110 and 138 can include one or more communication interfaces (e.g., wired and/or wireless) for communicating test results and/or measurements to other devices such as the medical system 120, the collaborative smart screen 102, and/or any other device.

The one or more sensors 112 and 134 can include any sensor device such as, for example, an infrared (IR) sensor, a biosensor, a tactile sensor, a pressure sensor, a respiratory sensor, a blood analyzer, a chemical sensor, an implantable sensor, a wearable sensor, a cataract sensor, a glucose meter, an activity sensor, a blood pressure sensor, a pulse oximeter, a heart rate sensor, a sleep sensor, a temperature sensor, a body composition analyzer, a stethoscope, and/or any other type of sensor. The one or more sensors 112 and 134 can include one or more communication interfaces (e.g., wired and/or wireless) for communicating test results and/or measurements to other devices such as the medical system 120, the collaborative smart screen 102, and/or any other device.

The one or more medical devices 114 and 140 can include any mechanical and/or electrical devices. For example, the one or more medical devices 114 and 140 can include a ventilator, a kidney dialysis machine, an insulin pump, a clinical bed, an anesthesia delivery machine, an oxygen concentrator, a surgical tool, a hearing test device, an ophthalmic testing device, a scope, a medicine delivery system, and/or any other medical device. The one or more medical devices 114 and 140 can include one or more communication interfaces (e.g., wired and/or wireless) for communicating test results and/or measurements to other devices such as the medical system 120, the collaborative smart screen 102, and/or any other device.

The one or more computing devices 116 and one or more client devices 132 can include a laptop computer, a desktop computer, a tablet computer, a mobile phone, an Internet-of-Things (IoT) device, a smart wearable device (e.g., a smart watch, an augmented reality device, a head-mounted display device, a smart ring, a smart meter, an activity tracker, etc.), a server, and/or any other computing device. The one or more computing devices 116 and one or more client devices 132 can include one or more communication interfaces (e.g., wired and/or wireless) for communicating test results and/or measurements to other devices such as the medical system 120, the collaborative smart screen 102, and/or any other device.

The third-party medical systems 136 can include one or more computing systems associated with one or more third parties and/or entities such as, for example, a hospital, a clinic, a doctor's office, a laboratory, a health insurance company, a health provider, etc. The third-party medical systems 136 can store, collect, track, and/or monitor health information associated with patients. For example, the third-party medical systems 136 can store and/or maintain health records, health data, medical orders, prescriptions, health metrics, medical procedure data, health statistics, health plans, patient data, etc. The third-party medical systems 136 can include one or more communication interfaces (e.g., wired and/or wireless) for communicating test results and/or measurements to other devices such as the medical system 120, the collaborative smart screen 102, and/or any other device.

As previously noted, the system 120 environment in FIG. 1 can be used to provide medical care, consultations, and/or related services. As used herein, a “consultation” can include an onsite consultation, a remote consultation (e.g., telemedicine, etc.), or a hybrid onsite and remote consultation where one or more participants are located on site and one or more participants are located remotely. In some examples, the medical system 120, any of the set of devices 102-116 in the medical care site 100, and/or any of the set of devices 132-140 at the one or more offsite locations 130 can communicate and/or interconnect via a network 125, and can share patient and medical data. The network 125 can include one or more public and/or private networks such as, for example, one or more cloud networks, local area networks, wide area networks, virtual networks, service provider networks, core networks, datacenters, and/or the like. In some cases, the network 125 can represent the Internet. Data from any of the set of devices 102-116 in the medical care site 100, and/or any of the set of devices 132-140 at the one or more offsite locations 130 can be received by the medical system 120 via a peer-to-peer connection (e.g., wireless or wired) and/or via one or more networks (e.g., a wired and/or wireless local area network). For example, in some cases, some or all of devices 132-140 that are within an offsite location can interconnect and/or communicate via one or more wireless connections and/or protocols (e.g., WIFI, Bluetooth, near-field communications, facsimile, etc.) and/or via a LAN. Upon receipt of the medical data, the data can be processed by the medical system 120 including utilizing optical character recognition (OCR) and a transcription user interface (UI). In some implementations, the medical system 120 can further receive updates (e.g., updated data) from any of the set of devices 102-116 in the medical care site 100, and/or any of the set of devices 132-140 at the one or more offsite locations 130 via the network 125.

In some examples, one or more of the devices 102-116 in the medical care site 100 can communicate and/or interconnect with one or more other devices 102-116 in the medical care site 100 directly via a peer-to-peer connection (e.g., wireless or wired) and/or via one or more networks (e.g., a wired and/or wireless local area network) on the medical care site 100. For example, in some cases, some or all of the devices 102-116 in the medical care site 100 can interconnect and/or communicate via one or more wireless connections and/or protocols (e.g., WIFI, Bluetooth, near-field communications, etc.) and/or via a local area network (LAN).

Similarly, in some examples, one or more of the devices 132-140 at the one or more offsite locations 130 can communicate and/or interconnect with one or more other devices 132-140 at the one or more offsite locations 130 directly via a peer-to-peer connection (e.g., wireless or wired) and/or via one or more networks (e.g., a wired and/or wireless local area network) at the one or more offsite locations 130. For example, in some cases, some or all of devices 132-140 that are within an offsite location can interconnect and/or communicate via one or more wireless connections and/or protocols (e.g., WIFI, Bluetooth, near-field communications, etc.) and/or via a LAN.

In some examples, the medical system 120 can collect data from one or more devices at the medical care site 100 (e.g., 102-116) and/or the one or more offsite locations 130 (e.g., 132-140). The medical system 120 can also provide data stored at the medical system 120 to one or more devices at the medical care site 100 (e.g., 102-116) and/or the one or more offsite locations 130 (e.g., 132-140).

Moreover, the collaborative smart screen 102 can send and/or receive data to/from the medical system 120 and devices 104-116 at the medical care site 100. In some cases, the collaborative smart screen 102 can also send and/or receive data to/from one or more of the devices 132-140 at the one or more offsite locations 130. For example, as further described herein, the collaborative smart screen 102 can collect data from the medical system 120 and/or any of the devices 104-116 at the medical care site 100. The collaborative smart screen 102 can use the collected data to present relevant medical and/or patient information on the collaborative smart screen 102 during a patient consultation at the medical care site 100.

In some cases, the collaborative smart screen 102 can dynamically collect, load, and/or display information during the patient consultation based on an action/task performed by the provider (e.g., a test, a measurement, an examination, a diagnosis, an input, an interaction with the patient, a question, a speech recognized by the collaborative smart screen 102, etc.), a context associated with the consultation (e.g., a reason for the consultation, a current topic of the consultation, a test and/or procedure performed and/or discussed during the consultation, biometrics associated with the patient, a condition relevant to the consultation, relevant patient information, a diagnosis associated with the consultation, a task and/or action associated with the consultation, etc.), and/or any other contextually-relevant factor.

For example, while a medical condition is addressed/discussed during the patient consultation, the collaborative smart screen can display information about the patient and relevant to the medical condition. If, while addressing the medical condition, the provider performs a test or measurement using one or more of the devices 104-116 at the medical care site 100, the collaborative smart screen 102 can dynamically collect (e.g., via push and/or pull) and display data from the test or measurement. The collaborative smart screen 102 can collect the data from the one or more of the devices 104-116 and display such data while the patient and provider address/discuss the medical condition associated with the test or measurement. If the consultation subsequently shifts to a different topic, item and/or task, the collaborative smart screen 102 similarly can dynamically collect, load, and/or display information relevant to the patient and the different topic, item, and/or task.

This way, the collaborative smart screen 102 can dynamically and intelligently gather, load, and present information relevant to a current portion of the consultation (e.g., a current topic, action, comment, etc.) and/or the consultation as a whole. As any of the devices 104-116 are used by the provider to obtain relevant patient data (e.g., test results, biometrics, measurements, etc.) during the consultation, the collaborative smart screen 102 can obtain such data and use the data to update the information presented by the collaborative smart screen 102 during the consultation. The collaborative smart screen 102 can also collect, load, and/or display relevant information obtained from other devices, such as test results, health metrics, and/or medical records from third-party systems, health metrics (e.g., measurements, statistics, test results, journal data, logged data, etc.) from one or more client devices associated with the patient (e.g., a smart watch, a heart rate sensor, a blood pressure sensor, a blood sugar sensor, a sleep sensor, an activity sensor, an image sensor, a pulse oximeter, a temperature sensor, a calorie tracker, a continuous positive airway pressure device, etc.), and/or any other device.

In some cases, the collaborative smart screen 102 can use available and/or loaded information to guide a patient consultation. For example, the collaborative smart screen 102 can dynamically display suggestions, tasks, relevant and/or contextual data, health metrics, agenda items, action items, and/or any other information tailored to allow (and/or inform) a provider to provide medical decisions and/or take actions during and/or for a patient consultation.

As further described herein, the collaborative smart screen 102 can include artificial intelligence and/or machine learning engines for performing one or more speech, image, and/or data processing tasks. In some cases, the collaborative smart screen 102 can include a speech processing engine for analyzing and recognizing speech, and generating a transcription of recognized speech. The collaborative smart screen 102 can thus recognize, transcribe, and display speech and conversations during a patient consultation. In some cases, the collaborative smart screen 102 can also generate speech audio (e.g., via text-to-speech) to output audio instructions, suggestions, messages, notifications, and/or other utterances.

While the system environment in FIG. 1 is shown to include certain devices and components, one of ordinary skill will appreciate that the system environment can include more or fewer of the same and/or different devices and components than those shown in FIG. 1. For example, in some cases, the system environment can include more/less and/or different sensors, medical devices, computing devices, and/or any other systems than those shown in FIG. 1. The devices and components in FIG. 1 are merely illustrative examples provided for explanation purposes.

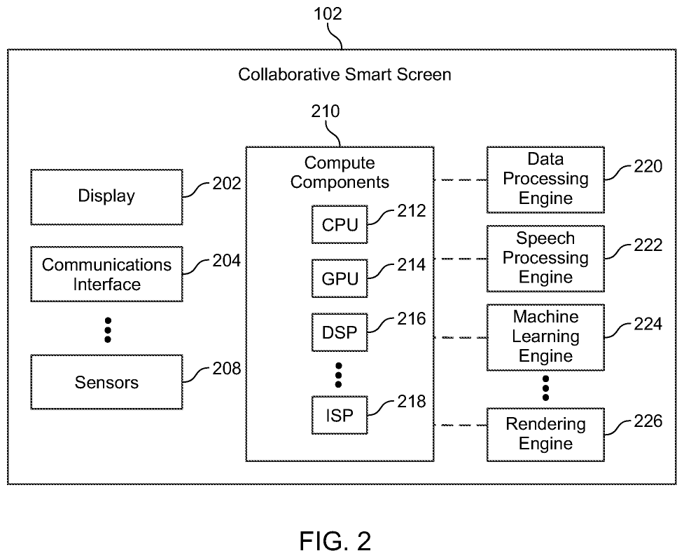

FIG. 2 is a diagram illustrating an example configuration of the collaborative smart screen 102. In this illustrative example, the collaborative smart screen 102 includes one or more displays 202, one or more communications interfaces 204 (e.g., wired and/or wireless), one or more sensors 208, compute components 210, a data processing engine 220, a speech processing engine 222, a machine learning engine 224, and a rendering engine 226. It should be noted that the components 202-226 shown in FIG. 2 are non-limiting examples provided for illustrative and explanation purposes, and other examples can include more, less, or different components than those shown in FIG. 2. For example, in some cases, the collaborative smart screen 102 can include one or more other sensors, one or more output devices, one or more input devices, one more other processing engines, one or more other hardware components, and/or one or more other software and/or hardware components that are not shown in FIG. 2. An example architecture and example hardware components that can be implemented by the collaborative smart screen 102 are further described below with respect to FIG. 7.

Moreover, references to any of the components (e.g., 202-226) of the collaborative smart screen 102 in the singular or plural form should not be interpreted as limiting the number of such components implemented by the collaborative smart screen 102 to one or more than one. For example, references to a display in the singular form should not be interpreted as limiting the number of displays implemented by the collaborative smart screen 102 to one. One of ordinary skill in the art will recognize that, for any of the components 202-226 shown in FIG. 2, the collaborative smart screen 102 can include only one of such component(s) or more than one of such component(s).

The collaborative smart screen 102 can be part of, or implemented by, a single computing device or multiple computing devices. In some examples, the collaborative smart screen 102 can be part of an electronic device (or devices) such as a display device, a computing device, etc.

In some implementations, the one or more displays 202, one or more communications interfaces 204, one or more sensors 208, compute components 210, data processing engine 220, speech processing engine 222, machine learning engine 224, and rendering engine 226 can be part of the same computing device. For example, in some cases, the one or more displays 202, one or more communications interfaces 204, one or more sensors 208, compute components 210, data processing engine 220, speech processing engine 222, machine learning engine 224, and rendering engine 226 can be integrated into a computing device. However, in some implementations, the one or more displays 202, one or more communications interfaces 204, one or more sensors 208, compute components 210, data processing engine 220, speech processing engine 222, machine learning engine 224, and rendering engine 226 can be part of two or more separate computing devices. For example, in some cases, some of the components 202-226 can be part of, or implemented by, one computing device and the remaining components can be part of, or implemented by, one or more other computing devices.

The one or more displays 202 can include any display device such as, for example, a computer screen, a television display, a touch screen, and the like. The one or more communication interfaces 204 can include any wired and/or wireless interfaces for communicating data with other devices. The one or more sensors 208 can include any sensor device such as, for example, an image or camera sensor, an audio sensor or microphone, a tactile sensor, a pressure sensor, a light sensor, a noise sensor, a motion sensor, a proximity sensor, a gyroscope, an accelerometer, a machine vision sensor, a speech recognition sensor, a shock sensor, a position sensor, etc.

The one or more compute components 210 can include, for example, a central processing unit (CPU) 212, a graphics processing unit (GPU) 214, a digital signal processor (DSP) 216, and/or an image signal processor (ISP) 218. The compute components 210 can perform various operations such as graphics rendering, data processing, networking operations, image enhancement, computer vision, extended reality (e.g., tracking, localization, pose estimation, mapping, content anchoring, content rendering, etc.), image/video processing, sensor processing, recognition (e.g., text recognition, facial recognition, object recognition, feature recognition, tracking or pattern recognition, scene recognition, speech recognition, gesture recognition, etc.), machine learning, filtering, and any of the various operations described herein.

In this example, the compute components 210 implement the data processing engine 220, speech processing engine 222, machine learning engine 224, and rendering engine. In other examples, the compute components 210 can also implement one or more other processing engines. The operations for the data processing engine 220, speech processing engine 222, machine learning engine 224, and rendering engine (and any other processing engines) can be implemented by any of the compute components 210. In one illustrative example, the operations of the rendering engine 226 can be implemented by the GPU 214, and the operations of the data processing engine 220, speech processing engine 222, and/or machine learning engine 224 can be implemented by the CPU 212, the DSP 216, and/or the ISP 218. In some cases, the compute components 210 can include other electronic circuits or hardware, computer software, firmware, or any combination thereof, to perform any of the various operations described herein.

The data processing engine 220, speech processing engine 222, machine learning engine 224, and rendering engine 226 can perform respective operations based on data stored by the collaborative smart screen 102, obtained from the one or more sensors 208, and/or received from the medical system 120, one or more of the devices 104-116 at the medical care site 100, and/or one or more of the devices 132-140 at the one or more offsite locations 130. In some examples, the data processing engine 220 can process and/or analyze digital, image, and/or video data to perform calculations, generate suggestions, implement workflows, modify computer content, generate outputs, etc.

The speech processing engine 222 can process and recognize speech utterances and generate transcripts corresponding to the recognized speech. In some cases, the speech processing engine 222 can also convert text to speech to generate speech outputs based on text. In some examples, the speech processing engine 222 can include a natural language processing (NLP) system.

The rendering engine 226 can process and render data for presentation by the display 202. Moreover, the machine learning engine 224 can implement one or more neural networks and/or machine learning models to perform one or more machine learning tasks. Non-limiting examples of machine learning tasks can include computer vision, image processing, medical diagnosis, NLP, recommender systems, pattern and/or sequence analysis, health monitoring, user behavior analytics, pattern recognition, decision making, health metrics analytics, medical testing analytics, information retrieval, optimization, and the like.

In some examples, the machine learning engine 224 can be separate from the data processing engine 220, the speech processing engine 222, and/or the rendering engine 226. In other examples, the machine learning engine 224 can be part of and/or implemented by the data processing engine 220, the speech processing engine 222, and/or the rendering engine 226.

While the collaborative smart screen 102 is shown to include certain components, one of ordinary skill will appreciate that the collaborative smart screen 102 can include more or fewer components than those shown in FIG. 2. For example, the collaborative smart screen 102 can also include, in some instances, one or more memory devices (e.g., RAM, ROM, cache, and/or the like), one or more other networking interfaces (e.g., wired and/or wireless communications interfaces and the like), one or more output and/or input devices, and/or other hardware or processing devices that are not shown in FIG. 2. An illustrative example of a computing device and hardware components that can be implemented with the collaborative smart screen 102 is described below with respect to FIG. 7.

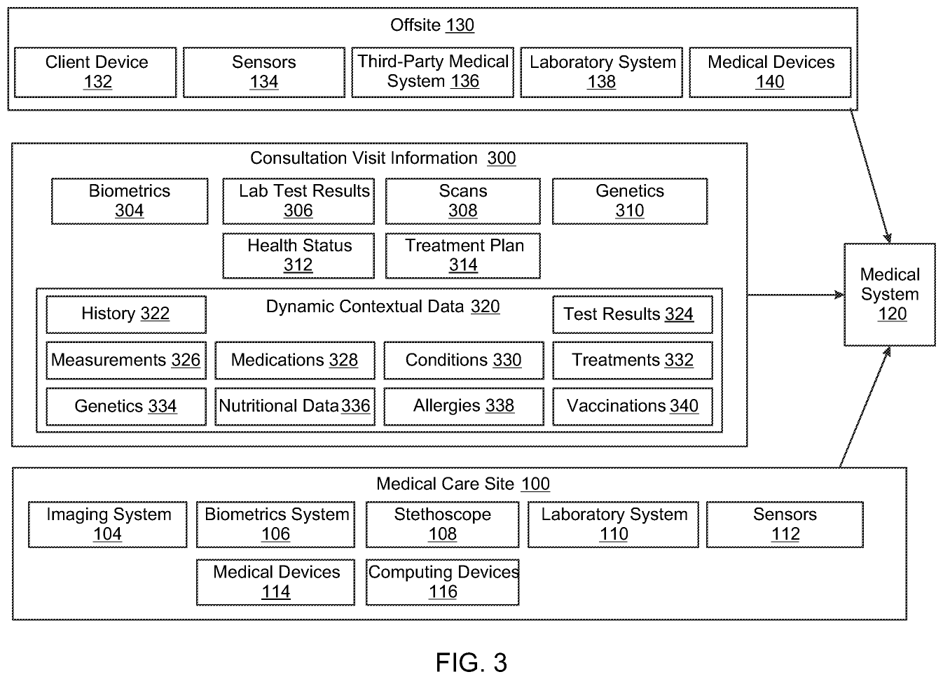

FIG. 3 is a diagram illustrating an example configuration of the medical system 120 receiving data from offsite 130, a consultation visit 300, and the medical care site 100. The medical system 120 can further receive data from member device applications (“apps”) and third-party consultations of the patient. In some examples, the data/information from the consultation visit 300 can include data generated during the patient consultation. For example, the consultation data 300 can include a transcription of some or all discussions/speech during the consultation, notes generated during the consultation, orders generated during the consultation, prescriptions created during the consultation, etc.

In some instances, the medical system 120 can dynamically load data based on an action(s) taken by the provider (e.g., an examination conducted by the provider, a test performed by the provider, a decision made by the provider, a procedure performed by the provider, a question or comment by the provider, an order/prescription issued by the provider, etc.), a topic addressed/discussed during the consultation, an issue raised during the consultation, a purpose of the consultation, information provided by the patient during the consultation, and/or any relevant event and/or circumstances. The medical system 120 can dynamically provide suggestions based on a current context and/or information associated with the patient. The suggestions can include, for example and without limitation, actions to take (e.g., orders, prescriptions, tests, procedures, examinations, referrals, treatments, questions, etc.), topics/items to address (e.g., medical issues, conditions, symptoms, diagnosis, treatment, plans, tests, etc.), issues and/or information to examine and/or verify, and/or any other activity and/or information relevant to the consultation.

In FIG. 3, the medical system 120 is shown receiving data from offsite 130, the consultation visit 300, and the medical care site 100. The consultation visit information 300 in this example includes data from dynamic contextual data 320, a biometrics interface element 304, a testing interface element 306, a scans interface element 308, a genetics interface element 310, a health status interface element 312, and a treatment plan interface element 314.

In some examples, the medical system 120 can provide customized medical treatment based on information gathered at the consultation by providing tasks, topics, and/or associated information based on patient data (e.g., offsite 130, the consultation visit information 300, and the medical care site 100). For example, the biometrics interface element 304, testing interface element 306, scans interface element 308, genetics interface element 310, health status interface element 312, and treatment plan interface element 314 can be utilized by the medical system 120 to cover (e.g., review, obtain, request, and/or consider) patient biometrics, tests, scans, genetics, health status, treatment plan(s), and any additional tasks. The provider can cover each item (e.g., 304-314) to the medical system 120 to provide a thorough, customized, and/or successful medical treatment. As further described below, information in the consultation visit 300 can be updated based on past and current data and/or decisions obtained while addressing/covering that item and/or another item by the medical system 120.

The biometrics interface element 304 can represent biometrics data associated with the patient, an action item for biometrics, and/or a selectable interface object for accessing (and/or navigating to) biometrics data associated with the patient. For example, in some cases, the biometrics interface element 304 can represent biometrics information of the patient presented in the consultation visit 300. In other cases, the biometrics interface element 304 can be a label or header representing an action item for biometrics to indicate that the provider should consider, verify, measure, cover, and/or update biometrics of the patient in preparation of a customized medical treatment. In yet other cases, the biometrics interface element 304 can represent a menu for accessing biometrics of the patient. The biometrics data can include health metrics collected and/or monitored for the patient such as, for example, blood pressure, heart rate, glucose levels, body temperature, body weight, pulse oximetry, etc.

The testing interface element 306 can represent test data associated with the patient, an action item for testing, and/or a selectable interface object for accessing (and/or navigating to) test data associated with the patient. For example, in some cases, the testing interface element 306 can represent test data of the patient (e.g., previous and/or current test results) presented in the consultation visit 300. In other cases, the testing interface element 306 can be a label or header representing an action item for testing to indicate that the provider should consider, verify, cover, perform and/or update tests of the patient. In yet other cases, the testing interface element 306 can represent a menu for accessing test data of the patient from the medical system 120. The test data can include test results collected and/or monitored for the patient such as, for example, blood tests, biopsies, saliva tests, stool tests, and/or any other medical tests.

The scans interface element 308 can represent scans associated with the patient, an action item for scans, and/or a selectable interface object for accessing (and/or navigating to) scans associated with the patient. The scans can include any scans and/or imaging results collected and/or monitored for the patient such as, for example, body scans, skin scans, CT scans, MRIs, PET scans, and/or any other medical scans.

The genetics interface element 310 can represent genetics data associated with the patient, an action item for genetics data, and/or a selectable interface object for accessing (and/or navigating to) genetics associated with the patient. The genetics data can include any genetic information, tests, and/or analysis obtained and/or monitored for the patient. Genetics information can help the patient and provider understand long-term health risks, health strategies, health insights, etc., and can be used to tailor and/or optimize health plans, treatments, and/or strategies for the patient.

The health status interface element 312 can represent health status information associated with the patient, an action item for health status, and/or a selectable interface object for accessing (and/or navigating to) health status information associated with the patient. The health status can include any information about the overall health and/or wellbeing of the patient such as, for example, health metrics, risks, conditions, normal and/or abnormal health parameters, etc.

The treatment plan interface element 314 can represent treatment plan data associated with the patient, an action item for a treatment plan, and/or a selectable interface object for accessing (and/or navigating to) treatment plan data associated with the patient. The treatment plan data can include one or more treatment plans (and associated statistics). A treatment plan can include, for example, diet, medications, procedures, lifestyle habits, care instructions, etc. The treatment plan data can also include past medical treatment plans to further determine whether the past medical treatment plan was successful.

The dynamic contextual data 320 of the consultation visit information 300 can include data dynamically loaded, displayed, and/or updated based on a current context of the consultation. For example, the dynamic contextual data 320 can include medical history information 322, test results 324, measurements 326, medications 328, conditions 330, treatments 322, genetics 334, nutritional data 336, allergies 338, vaccinations 340, etc., that is/are relevant to a current agenda item (e.g., 304-314) being covered, a current topic being covered, a current action being performed (e.g., a current test, examination, procedure, etc.), a current decision being made by the provider, and/or any other current circumstances.

The medical history information 322 can include past and present medical history of the patient measured from home, offsite 130, consultation visit 300, or medical care site 100. The medical history information 322 can also include measurements taken by the patient or a healthcare professional (e.g., temperature, blood pressure, scan of skin, etc.).

The test results 324 can include test results from medical tests conducted at home, offsite 130, consultation visit 300, or medical care site 100. The medical tests can be performed by the patient or a healthcare professional, and the test results 324 provided to the medical system 120.

The measurements 326 can include measurements of body parts, questionable formations, temperature, or any other measurement suitable for the intended purpose and understood by a person of ordinary skill in the art. The measurements 326 can be performed by the patient or a healthcare professional, and the test results 324 provided to the medical system 120.

The medications 328 can include past and present medications taken by the patient and/or prescribed by a healthcare professional. The medications 328 can also include legal and illegal substances or drugs taken by the patient. This information may be important to the medical system 120 so that a contradicting prescription or procedure is not presented to the patient.

The conditions 330 can include past and present conditions of the patient such as body temperature, mental awareness, surgeries, stamina, blood sugar levels, etc. The conditions 330 can be provided by the patient, assessed by the sensors 112, 134, or assessed by the healthcare professional at the medical care site 100, the offsite location 130, or the consultation visit 300.

The treatments 332 can include past treatments that were completed or not fully completed by the patient such as medical routines, exercises, water intake amounts, etc. The treatments 332 can also include treatments researched by the patient through self-diagnosis (e.g., WebMD) and treatments tailored for the patient by a healthcare professional.

The genetics 334 can include genetic results from self-administered genetic tests or genetic tests conducted at the consultation visit 300 or the medical are site 100. The genetics 334 can also include past and present genetic test results and makeup from relatives of the patient to provide a better understanding of the patient's overall health.

The nutritional data 336 can include past and present nutritional data prescribed by the patient such as a daily nutritional routine, caloric intact amount, balanced diet regime, etc. The nutritional data 336 can also include past and present nutritional data prescribed by a healthcare professional at the medical care site 100, the offsite location 130, and the consultation visit 300.

In some examples, the dynamic contextual data 320, the biometrics 304, the lab test results 306, the scans 308, the genetics 310, the health status 312, and the treatment plans 314 of the consultation visit information 300 can be dynamically based on data associated with the patient and/or the consultation received (e.g., wirelessly and/or via a wired network connection) from the medical system 120, one or more systems (e.g., 104-116) in the medical care site 100 and/or one or more devices (e.g., 132-140) in the one or more offsite locations 130. For example, before and/or during the consultation, the medical system 120 can receive data relevant from the patient and consultation such as a medical record of the patient. The medical system 120 can use the data received to determine a customized medical treatment for the patient.

In some cases, new and updated data can be received by the medical system 120 and/or one or more systems. For example, the provider can use the sensors 112 to measure biometrics of the patient, such as a heart rate, blood pressure, weight, blood glucose levels, oxygen levels, etc. The medical system 120 can then receive (e.g., via a wired and/or wireless transmission) the newly measured biometrics from the sensors 112, and update the customized medical treatment based on updated data from offsite 130, the consultation visit 300, and the medical care site 100.

If the provider uses the one or more imaging systems 104 to obtain a scan (e.g., a body scan, a skin scan, a CT scan, etc.) for the patient during the consultation, the medical system 120 can receive the scan from the one or more imaging systems 104 and similarly update the customized medical treatment. In this way, any data (e.g., measurements, outputs, results, etc.) collected by the provider using any of the devices 104-116 in the medical care site 100 can be dynamically loaded and updated on the medical system 120 to provide continuously updating customized medical treatments.

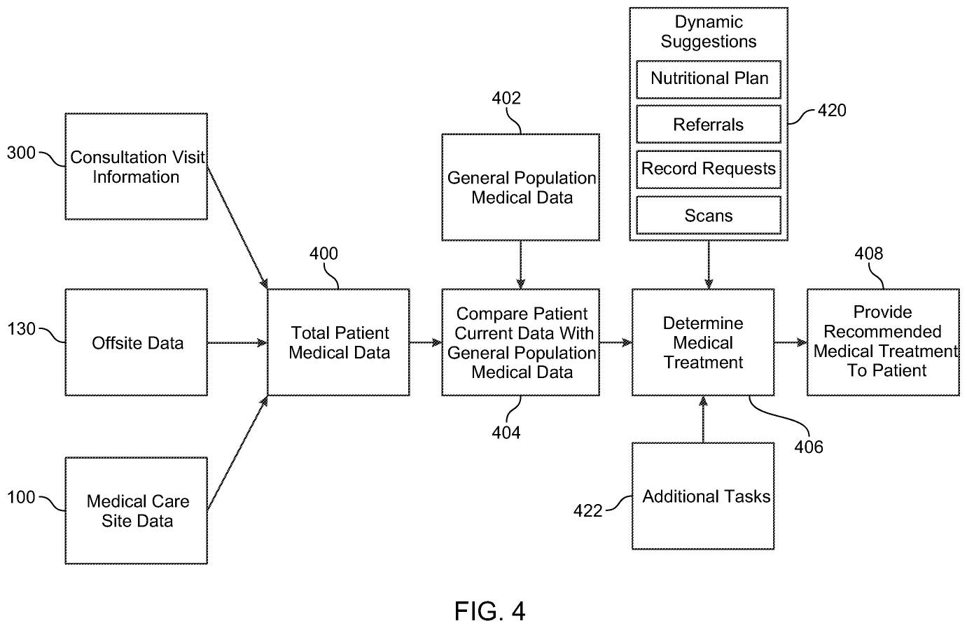

FIG. 4 is a diagram illustrating an example process of providing a customized medical treatment 408 by the medical system 120. In this example, a total patient medical data 400 can include data from the consultation visit 300, from offsite 130, and the medical care site 100. For example, the total patient medical data 400 can include sex, date of birth, pre-existing medical conditions, genetic makeup, body scans, blood type, blood pressure, heart rate, etc. to provide a comprehensive medical treatment where necessary or recommended.

The combination of all or some of the data from the consultation visit 300, from offsite 130, and the medical care site 100 can further be utilized and compared 404 to medical data of the general population 402 such that the medical system 120 can determine whether treatment is necessary or wanted for a given condition. The general population medical data 402 can be received by the medical system 120 from hospitals, institutions, governments, third party companies, etc. The general population medical data 402 can include information regarding all or some variants of diseases, conditions, ailments, symptoms, etc. such that the comparison 404 can quickly determine whether the current patient is at risk for any given numbers of conditions.

For example, a patient may have a skin formation that is of concern. The medical system 120 can scan, dissect, measure, and photograph the skin formation and compare it to skin formations of similar size, color, duration, complexity, etc. The medical system 120 can also take into account the patient's weight, sex, eating habits, outdoor activities, etc. to find a group of the general population that matches or has a similar lifestyle to the patient to provide more accurate results and recommendations. Doctors are only able to make connections between cases based on their knowledge and experience. The medical system 120 can scour millions upon millions of records, scans, photos, prognoses, measurements, etc. in a very short period of time and provide closer matches based on life style, eating habits, race, sex, etc.

The medical system 120 can further receive dynamic suggestions 420 to determine which medical treatment is most appropriate for the patient. The dynamic suggestions 420 in can include suggestions dynamically generated, displayed, and/or updated based on information gathered from the consultation visit 300. For example, the dynamic suggestions 420 can include nutritionals plans, referrals, record requests, scans, suggested tests, measurements, diet plans, medications, treatments, procedures, examinations, orders, actions, etc. In some examples, such suggestions can be generated, displayed, and/or updated based on the biometrics 304, the lab test results 306, the scans 308, the genetics 310, the health status 312, and the treatment plan 314 regarding a current topic being covered, a current action being performed (e.g., a current test, examination, procedure, etc.), a current decision being made by the provider, patient data previously obtained and/or determined, patient data obtained and/or determined during the consultation, and/or any other relevant information.

In some examples, the medical system 120 may further receive information regarding additional tasks 422 that can represent data associated with additional tasks for the consultation, an action item for additional, and/or a selectable interface object for accessing (and/or navigating to) additional tasks associated with the patient. The additional tasks 422 can include any other tasks not covered in the consultation 300 and/or resulting from other items covered elsewhere, such as referrals, record requests, chat threads with members or patients, additional tests, topics, treatments, orders, medications, examinations, protocols, instructions, procedures, checks, decisions, etc.

Upon receiving data regarding the comparison between the patient's current data 400 and the general population medical data 402, the dynamic suggestions 420, and the additional tasks 422, the medical system can analyze all or some of this data to determine whether a notable condition is present, and if so, which appropriate medical treatment 406 is necessary or recommended at this time. Upon selection of the determined medical treatment 406, the medical system 120 can provide the determined medical treatment 406 to the patient 408 for immediate action.

For example, the determination of medical treatment 406 can be based on all or some of the information and data received from the consultation visit 300, from offsite 130, the medical care site 100, the general population 402, the dynamic suggestions 420, and the additional tasks 422. Based on all or some of these factors, the medical system 120 can determine if the patient has a condition worth noting and pursuing a customized treatment plan. For example, all skin conditions are not treated by the same method. Based on the type, size, growth rate, patient weight, patient diet, etc., the detected skin condition should be treated accordingly in an individualized manner.

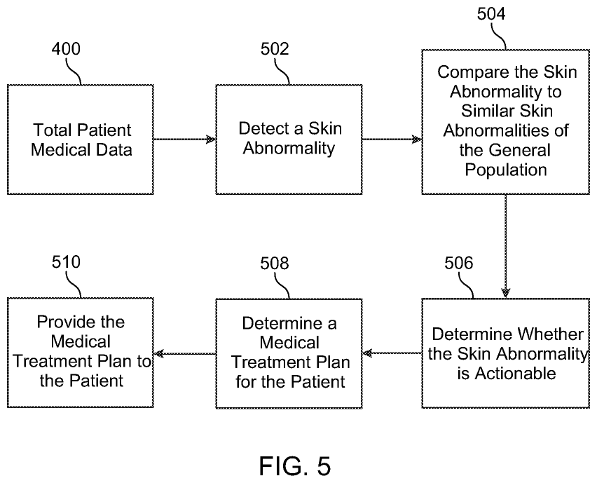

FIG. 5 is a diagram illustrating example process of providing a customized skin condition treatment recommendation. In some examples, the total patient medical data 400 of the medical system 120 can be utilized to detect a skin abnormality 502. For example, scans of the patient's skin can be received by the medical system 120 from the sensors 134 of the offsite location 130, the sensors 112 of the medical care site 100, and the scans 308 from the consultation visit 300. The medical system 120 can also take into consideration the genetics 310 of the patients and pre-existing conditions from the dynamic contextual data 320 to determine whether a skin abnormality is of concern. For example, if the patient has a long family history of skin disease, then a rather small and insignificant skin formation may be cause for alarm. Particular living conditions, dietary plans, and/or nutritional intake may also affect skin conditions and can be taken into consideration by the medical system 120 in determining whether the skin formation is a skin abnormality that warrants a customized medical treatment.

The detected skin abnormality 502 can further be compared to similar skin conditions and abnormalities of the general population 504. For example, the medical system 120 can receive data of past patients from hospitals, institutions, governments, third party companies, etc. that include skin formations of similar height, width, weight, color, length, growth rate, density, etc. The medical system 120 can further include receiving data of past patients with similar skin conditions who are of similar weight, size, sex, race, age, genetic makeup, etc. of the patient to provide a more individualized medical treatment. Based on this information, the medical system 120 can determine whether the skin abnormality is actionable 506. For example, based on the data received regarding similar skin abnormalities from past patients of similar body structure, genetic makeup, and nutritional lifestyle, the medical system 120 can determine whether to take action in treating the detected skin abnormality 502.

Upon determining that the skin abnormality is actionable 506, the medical system 120 can further receive data regarding similar skin abnormalities from past patients of similar body structure, genetic makeup, and nutritional lifestyle, their eventual outcome, and which past medical treatments concluded with a positive outcome. For example, the medical system 120 can receive data regarding medical treatment plans that were provided for the similar skin abnormalities found in the general population 504. The outcome of the medical treatment plans provided to the general population can also be taken into consideration by the medical system 120 in determining which medical treatment is best for the current patient.

In some examples, based on the effectiveness and timeline of the medical treatments from the general population regarding the detected skin abnormality 502 of the current patient, the medical system 120 can select the medical treatment that is most appropriate for the current patient, which can then be provided to the current patient 510. For example, if a skin abnormality is found on a male, who is 160 pounds, 35 years old, has a genetic makeup with a propensity of skin disease, and eats an unbalanced diet, the medical system 120 can take all or some of these factors into consideration when comparing the male's skin abnormality with skin conditions from the general population that have the same type, size, density, and color as the male's skin abnormality. By analyzing the patient's total medical data 400 and comparing the same to the general population of similar body structure, age, genetic makeup, etc., the medical system 120 can provide a customized medical treatment for any ailment or condition found at home (e.g., offsite 130), a consultation visit 300, or medical care site 100.

Having disclosed example systems, components and concepts, the disclosure now turns to the example method 600 for providing customized medical treatment, as shown in FIG. 6. The steps outlined herein are non-limiting examples provided for illustration purposes, and can be implemented in any combination thereof, including combinations that exclude, add, or modify certain steps.

At block 602, the method 600 can include receiving, at a medical system, patient data associated with a patient. In some examples, the patient data associated with the patient can be a consultation action (e.g., a test, an examination, a health metric measurement, a scan, a procedure, a treatment, an order, a prescription, a screening, a physical, etc.) determined based on a medical record of the patient, patient information collected during the patient consultation, and/or one or more health metrics (e.g., test results, biometrics, scans, examination results, etc.) generated/obtained during the patient consultation.

In some examples, the patient data associated with the patient can include actions such as performing a medical test, performing a medical examination, and/or measuring a health metric via one or more medical devices (e.g., 104-116) at the medical care site. In some cases, the medical test can include a blood test, a scan, collecting and analyzing a specimen (e.g., blood, saliva, stool, a skin sample, etc.) from the patient, a medical assessment, a genetic test, and/or a breathing test. In some cases, the health metric can include a blood pressure, blood glucose levels, a pulse, a body temperature, and/or a body weight.

In some cases, at least part of the patient data is received from a client device (e.g., 132) associated with the patient and/or one or more sensors (e.g., 104, 106, 108, 112, etc.) at the medical care site. In some examples, the client device can include a smart phone and/or a smart wearable device (e.g., a smart watch, an activity tracker, a smart ring, a portable sensor, a pulse oximeter, a blood pressure monitor, a sleep monitor, etc.), and the one or more sensors can include a wireless blood pressure sensor, a wireless heart rate sensor, a wireless body temperature sensor, a wireless pulse oximeter, a stethoscope, and/or an imaging sensor (e.g., a scanner, a camera, etc.).

At block 604, the method 600 can include detecting, at the medical system, a health condition of the patient based on the patient data associated with the patient. In some examples, a portion of patient data can include a patient health status, information from a patient medical record, measurements and/or metrics collected through a previous consultation action, etc.

At block 606, the method 600 can include comparing, at the medical system, the patient data and the health condition of the patient with similar health conditions of a general population. In some examples, the method 600 can include receiving the one or more measurements from one or more devices at the medical care site and dynamically updating the medical system based on the one or more measurements.

At block 608, the method 600 can include determining, at the medical system, whether the health condition of the patient is actionable based on the comparing of the patient data and the health condition of the patient with the similar health conditions of the general population.

At block 610, the method 600 can include providing, by the medical system, a customized medical treatment plan to the patient based on the determining of whether the health condition of the patient is actionable.

In some aspects, the method 600 can include receiving, from the one or more medical devices, a medical test result, a medical examination result and/or the health metric, and presenting the portion of patient data in response to receiving the medical test result, the medical examination result, and/or the health metric.

In some aspects, the method 600 can include determining an additional portion of patient data, and comparing the additional portion of patient data with the patient data and the health condition of the patient with the similar health conditions of the general population. In some examples, the additional portion of patient data can be based on a current context of the patient consultation and/or the patient data.

In some aspects, the method 600 can include providing, by the medical system, one or more workflow items determined at a patient consultation. In some examples, the one or more workflow items determined at a patient consultation, the one or more workflow items being based on at least one of the patient data and additional patient data collected during the patient consultation.

In some examples, the method 600 may be performed by one or more computing devices or apparatuses. In one illustrative example, the method 600 can be performed by the medical system 120 shown in FIG. 1 and/or one or more computing devices with the computing device architecture 700 shown in FIG. 7. In some cases, such a computing device or apparatus may include a processor, microprocessor, microcomputer, or other component of a device that is configured to carry out the steps of the method 600. In some examples, such computing device or apparatus may include one or more sensors configured to capture image data. For example, the computing device can include a smartphone, a head-mounted display, a mobile device, a display screen, or other suitable device. In some examples, such computing device or apparatus may include a display configured to display computer data and/or graphics. In some cases, such computing device may include a display for displaying digital data.

The components of the computing device can be implemented in circuitry. For example, the components can include and/or can be implemented using electronic circuits or other electronic hardware, which can include one or more programmable electronic circuits (e.g., microprocessors, graphics processing units (GPUs), digital signal processors (DSPs), central processing units (CPUs), and/or other suitable electronic circuits), and/or can include and/or be implemented using computer software, firmware, or any combination thereof, to perform the various operations described herein. The computing device may include a display (as an example of the output device or in addition to the output device), a network interface configured to communicate and/or receive the data, any combination thereof, and/or other component(s). The network interface may be configured to communicate and/or receive Internet Protocol (IP) based data or other type of data.

The method 600 is illustrated as a logical flow diagram, the operations of which represent a sequence of operations that can be implemented in hardware, computer instructions, or a combination thereof. In the context of computer instructions, the operations represent computer-executable instructions stored on one or more computer-readable storage media that, when executed by one or more processors, perform the recited operations. Generally, computer-executable instructions include routines, programs, objects, components, data structures, and the like that perform particular functions or implement particular data types. The order in which the operations are described is not intended to be construed as a limitation, and any number of the described operations can be combined in any order and/or in parallel to implement the processes.

Additionally, the method 600 may be performed under the control of one or more computer systems configured with executable instructions and may be implemented as code (e.g., executable instructions, one or more computer programs, or one or more applications) executing collectively on one or more processors, by hardware, or combinations thereof. As noted above, the code may be stored on a computer-readable or machine-readable storage medium, for example, in the form of a computer program comprising a plurality of instructions executable by one or more processors. The computer-readable or machine-readable storage medium may be non-transitory.

FIG. 7 illustrates an example computing device architecture 700 of an example computing device which can implement various techniques described herein. For example, the computing device architecture 700 can implement at least some portions of the medical system 120 shown in FIG. 1 and/or the collaborative smart screen 102 shown in FIGS. 1 and 2. The components of the computing device architecture 700 are shown in electrical communication with each other using a connection 705, such as a bus. The example computing device architecture 700 includes a processing unit (CPU or processor) 710 and a computing device connection 705 that couples various computing device components including the computing device memory 715, such as read only memory (ROM) 720 and random access memory (RAM) 725, to the processor 710.

The computing device architecture 700 can include a cache of high-speed memory connected directly with, in close proximity to, or integrated as part of the processor 710. The computing device architecture 700 can copy data from the memory 715 and/or the storage device 730 to the cache 712 for quick access by the processor 710. In this way, the cache can provide a performance boost that avoids processor 710 delays while waiting for data. These and other modules can control or be configured to control the processor 710 to perform various actions. Other computing device memory 715 may be available for use as well. The memory 715 can include multiple different types of memory with different performance characteristics. The processor 710 can include any general purpose processor and a hardware or software service stored in storage device 730 and configured to control the processor 710 as well as a special-purpose processor where software instructions are incorporated into the processor design. The processor 710 may be a self-contained system, containing multiple cores or processors, a bus, memory controller, cache, etc. A multi-core processor may be symmetric or asymmetric.

To enable user interaction with the computing device architecture 700, an input device 745 can represent any number of input mechanisms, such as a microphone for speech, a touch-sensitive screen for gesture or graphical input, keyboard, mouse, motion input, speech and so forth. An output device 735 can also be one or more of a number of output mechanisms known to those of skill in the art, such as a display, projector, television, speaker device. In some instances, multimodal computing devices can enable a user to provide multiple types of input to communicate with the computing device architecture 700. The communication interface 740 can generally govern and manage the user input and computing device output. There is no restriction on operating on any particular hardware arrangement and therefore the basic features here may easily be substituted for improved hardware or firmware arrangements as they are developed.

Storage device 730 is a non-volatile memory and can be a hard disk or other types of computer readable media which can store data that are accessible by a computer, such as magnetic cassettes, flash memory cards, solid state memory devices, digital versatile disks, cartridges, random access memories (RAMs) 725, read only memory (ROM) 720, and hybrids thereof. The storage device 730 can include software, code, firmware, etc., for controlling the processor 710. Other hardware or software modules are contemplated. The storage device 730 can be connected to the computing device connection 705. In one aspect, a hardware module that performs a particular function can include the software component stored in a computer-readable medium in connection with the necessary hardware components, such as the processor 710, connection 705, output device 735, and so forth, to carry out the function.

The term “computer-readable medium” includes, but is not limited to, portable or non-portable storage devices, optical storage devices, and various other mediums capable of storing, containing, or carrying instruction(s) and/or data. A computer-readable medium may include a non-transitory medium in which data can be stored and that does not include carrier waves and/or transitory electronic signals propagating wirelessly or over wired connections. Examples of a non-transitory medium may include, but are not limited to, a magnetic disk or tape, optical storage media such as compact disk (CD) or digital versatile disk (DVD), flash memory, memory or memory devices. A computer-readable medium may have stored thereon code and/or machine-executable instructions that may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, or the like.

In some embodiments the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Specific details are provided in the description above to provide a thorough understanding of the embodiments and examples provided herein. However, it will be understood by one of ordinary skill in the art that the embodiments may be practiced without these specific details. For clarity of explanation, in some instances the present technology may be presented as including individual functional blocks comprising devices, device components, steps or routines in a method embodied in software, or combinations of hardware and software. Additional components may be used other than those shown in the figures and/or described herein. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Individual embodiments may be described above as a process or method which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination can correspond to a return of the function to the calling function or the main function.

Processes and methods according to the above-described examples can be implemented using computer-executable instructions that are stored or otherwise available from computer-readable media. Such instructions can include, for example, instructions and data which cause or otherwise configure a general purpose computer, special purpose computer, or a processing device to perform a certain function or group of functions. Portions of computer resources used can be accessible over a network. The computer executable instructions may be, for example, binaries, intermediate format instructions such as assembly language, firmware, source code. Examples of computer-readable media that may be used to store instructions, information used, and/or information created during methods according to described examples include magnetic or optical disks, flash memory, USB devices provided with non-volatile memory, networked storage devices, and so on.

Devices implementing processes and methods according to these disclosures can include hardware, software, firmware, middleware, microcode, hardware description languages, or any combination thereof, and can take any of a variety of form factors. When implemented in software, firmware, middleware, or microcode, the program code or code segments to perform the necessary tasks (e.g., a computer-program product) may be stored in a computer-readable or machine-readable medium. A processor(s) may perform the necessary tasks. Typical examples of form factors include laptops, smart phones, mobile phones, tablet devices or other small form factor personal computers, personal digital assistants, rackmount devices, standalone devices, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are example means for providing the functions described in the disclosure.

In the foregoing description, aspects of the application are described with reference to specific embodiments thereof, but those skilled in the art will recognize that the application is not limited thereto. Thus, while illustrative embodiments of the application have been described in detail herein, it is to be understood that the inventive concepts may be otherwise variously embodied and employed, and that the appended claims are intended to be construed to include such variations, except as limited by the prior art. Various features and aspects of the above-described application may be used individually or jointly. Further, embodiments can be utilized in any number of environments and applications beyond those described herein without departing from the broader spirit and scope of the specification. The specification and drawings are, accordingly, to be regarded as illustrative rather than restrictive. For the purposes of illustration, methods were described in a particular order. It should be appreciated that in alternate embodiments, the methods may be performed in a different order than that described.

One of ordinary skill will appreciate that the less than (“<”) and greater than (“>”) symbols or terminology used herein can be replaced with less than or equal to (“≤”) and greater than or equal to (“≥”) symbols, respectively, without departing from the scope of this description.

Where components are described as being “configured to” perform certain operations, such configuration can be accomplished, for example, by designing electronic circuits or other hardware to perform the operation, by programming programmable electronic circuits (e.g., microprocessors, or other suitable electronic circuits) to perform the operation, or any combination thereof.

The phrase “coupled to” refers to any component that is physically connected to another component either directly or indirectly, and/or any component that is in communication with another component (e.g., connected to the other component over a wired or wireless connection, and/or other suitable communication interface) either directly or indirectly.

Claim language or other language reciting “at least one of” a set and/or “one or more” of a set indicates that one member of the set or multiple members of the set (in any combination) satisfy the claim. For example, claim language reciting “at least one of A and B” or “at least one of A or B” means A, B, or A and B. In another example, claim language reciting “at least one of A, B, and C” or “at least one of A, B, or C” means A, B, C, or A and B, or A and C, or B and C, or A and B and C. The language “at least one of” a set and/or “one or more” of a set does not limit the set to the items listed in the set. For example, claim language reciting “at least one of A and B” or “at least one of A or B” can mean A, B, or A and B, and can additionally include items not listed in the set of A and B.

The various illustrative logical blocks, modules, circuits, and algorithm steps described in connection with the examples disclosed herein may be implemented as electronic hardware, computer software, firmware, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present application.

The techniques described herein may also be implemented in electronic hardware, computer software, firmware, or any combination thereof. Such techniques may be implemented in any of a variety of devices such as general purposes computers, wireless communication device handsets, or integrated circuit devices having multiple uses including application in wireless communication device handsets and other devices. Any features described as modules or components may be implemented together in an integrated logic device or separately as discrete but interoperable logic devices. If implemented in software, the techniques may be realized at least in part by a computer-readable data storage medium comprising program code including instructions that, when executed, performs one or more of the methods, algorithms, and/or operations described above. The computer-readable data storage medium may form part of a computer program product, which may include packaging materials. The computer-readable medium may comprise memory or data storage media, such as random access memory (RAM) such as synchronous dynamic random access memory (SDRAM), read-only memory (ROM), non-volatile random access memory (NVRAM), electrically erasable programmable read-only memory (EEPROM), FLASH memory, magnetic or optical data storage media, and the like. The techniques additionally, or alternatively, may be realized at least in part by a computer-readable communication medium that carries or communicates program code in the form of instructions or data structures and that can be accessed, read, and/or executed by a computer, such as propagated signals or waves.

The program code may be executed by a processor, which may include one or more processors, such as one or more digital signal processors (DSPs), general purpose microprocessors, an application specific integrated circuits (ASICs), field programmable logic arrays (FPGAs), or other equivalent integrated or discrete logic circuitry. Such a processor may be configured to perform any of the techniques described in this disclosure. A general purpose processor may be a microprocessor; but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Accordingly, the term “processor,” as used herein may refer to any of the foregoing structure, any combination of the foregoing structure, or any other structure or apparatus suitable for implementation of the techniques described herein.

What is claimed is:

1. A method comprising:
receiving, at a medical system, patient data associated with a patient;
detecting, at the medical system, a health condition of the patient based on the patient data associated with the patient;
comparing, at the medical system, the patient data and the health condition of the patient with similar health conditions of a general population;
determining, at the medical system, whether the health condition of the patient is actionable based on the comparing of the patient data and the health condition of the patient with the similar health conditions of the general population; and
providing, by the medical system, a customized medical treatment plan to the patient based on the determining of whether the health condition of the patient is actionable.

2. The method of claim 1, wherein the patient data associated with the patient comprises at least one of performing a medical test, performing a medical examination, and measuring a health metric via one or more medical devices.

3. The method of claim 2, wherein the medical test comprises at least one of a blood test, a scan, collecting and analyzing a specimen from the patient, a medical assessment, a genetic test, and a breathing test.

4. The method of claim 2, wherein the health metric comprises at least one of blood pressure, blood glucose levels, a pulse, a body temperature, and a body weight.

5. The method of claim 1, further comprising:
determining an additional portion of patient data, the additional portion of patient data being based on a current context of a patient consultation; and
comparing the additional portion of patient data with the patient data and the health condition of the patient with the similar health conditions of the general population.

6. The method of claim 1, wherein at least part of the patient data is received from at least one of a client device associated with the patient and one or more sensors at a medical care site,
wherein the client device comprises at least one of a smart phone and a smart wearable device, and
wherein the one or more sensors comprise at least one of a wireless blood pressure sensor, a wireless heart rate sensor, a wireless body temperature sensor, a wireless pulse oximeter, a stethoscope, and an imaging sensor.

7. The method of claim 1, further comprising:
providing, by the medical system, one or more workflow items determined at a patient consultation, the one or more workflow items being based on at least one of the patient data and additional patient data collected during the patient consultation.

8. A system comprising:
memory; and
one or more processors coupled to the memory, the one or more processors being configured to:
receive, at a medical system, patient data associated with a patient;
detect, at the medical system, a health condition of the patient based on the patient data associated with the patient;
compare, at the medical system, the patient data and the health condition of the patient with similar health conditions of a general population;
determine, at the medical system, whether the health condition of the patient is actionable based on the comparison of the patient data and the health condition of the patient with the similar health conditions of the general population; and
provide, by the medical system, a customized medical treatment plan to the patient based on the determination of whether the health condition of the patient is actionable.

9. The system of claim 8, wherein the patient data associated with the patient comprises at least one of performing a medical test, performing a medical examination, and measuring a health metric via one or more medical devices.

10. The system of claim 9, wherein the medical test comprises at least one of a blood test, a scan, collecting and analyzing a specimen from the patient, a medical assessment, a genetic test, and a breathing test.

11. The system of claim 9, wherein the health metric comprises at least one of blood pressure, blood glucose levels, a pulse, a body temperature, and a body weight.

12. The system of claim 8, wherein the one or more processors being configured to:
determine an additional portion of patient data, the additional portion of patient data being based on a current context of a patient consultation; and
compare the additional portion of patient data with the patient data and the health condition of the patient with the similar health conditions of the general population.

13. The system of claim 8, wherein at least part of the patient data is received from at least one of a client device associated with the patient and one or more sensors at a medical care site,
wherein the client device comprises at least one of a smart phone and a smart wearable device, and
wherein the one or more sensors comprise at least one of a wireless blood pressure sensor, a wireless heart rate sensor, a wireless body temperature sensor, a wireless pulse oximeter, a stethoscope, and an imaging sensor.

14. The system of claim 8, wherein the one or more processors being configured to:
provide, by the medical system, one or more workflow items determined at a patient consultation, the one or more workflow items being based on at least one of the patient data and additional patient data collected during the patient consultation.

15. A non-transitory computer-readable storage medium comprising instructions stored thereon which, when executed by one or more processors, cause the one or more processors to:
receive, at a medical system, patient data associated with a patient;
detect, at the medical system, a health condition of the patient based on the patient data associated with the patient;
compare, at the medical system, the patient data and the health condition of the patient with similar health conditions of a general population;
determine, at the medical system, whether the health condition of the patient is actionable based on the comparing of the patient data and the health condition of the patient with the similar health conditions of the general population; and
provide, by the medical system, a customized medical treatment plan to the patient based on the determining of whether the health condition of the patient is actionable.

16. The non-transitory computer-readable storage medium of claim 15, wherein the patient data associated with the patient comprises at least one of performing a medical test, performing a medical examination, and measuring a health metric via one or more medical devices.

17. The non-transitory computer-readable storage medium of claim 16, wherein the medical test comprises at least one of a blood test, a scan, collecting and analyzing a specimen from the patient, a medical assessment, a genetic test, and a breathing test.

18. The non-transitory computer-readable storage medium of claim 16, wherein the health metric comprises at least one of blood pressure, blood glucose levels, a pulse, a body temperature, and a body weight.

19. The non-transitory computer-readable storage medium of claim 15, wherein the instructions stored thereon which, when executed by the one or more processors, cause the one or more processors to:
determine an additional portion of patient data, the additional portion of patient data being based on a current context of a patient consultation;
compare the additional portion of patient data with the patient data and the health condition of the patient with the similar health conditions of the general population; and
provide one or more workflow items determined at the patient consultation, the one or more workflow items being based on at least one of the patient data and the additional portion of patient data collected during the patient consultation.

20. The non-transitory computer-readable storage medium of claim 15, wherein at least part of the patient data is received from at least one of a client device associated with the patient and one or more sensors at a medical care site,
wherein the client device comprises at least one of a smart phone and a smart wearable device, and
wherein the one or more sensors comprise at least one of a wireless blood pressure sensor, a wireless heart rate sensor, a wireless body temperature sensor, a wireless pulse oximeter, a stethoscope, and an imaging sensor.
